(19) United States
(12) Patent Application Publication
(10) Pub. No.: US 2026/0207045 A1
(43) Pub. Date: Jul. 23, 2026

(54) SYSTEM FOR CHARACTERIZING CATARACT DENSITY

(71) Applicant: Alcon Inc., Fribourg (CH)

(72) Inventors: George Hunter Pettit, Fort Worth, TX (US); John Alfred Campin, St. Cloud, FL (US); Joseph Weatherbee, San Clemente, CA (US); Ramesh Sarangapani, Coppell, TX (US)

(21) Appl. No.: 19/448,198

(22) Filed: Jan. 14, 2026

Related U.S. Application Data
(60) Provisional application No. 63/747,705, filed on Jan. 21, 2025.

(51) Int. Cl. A61B 3/00; A61B 3/10; A61B 3/12; A61B 34/20; A61F 9/007; G16H 40/63; A61B 17/00; A61B 17/32

(52) U.S. Cl. CPC A61B 3/0025; A61B 3/102; A61B 3/1225; A61B 34/20; A61F 9/00745; G16H 40/63; A61B 2017/00057; A61B 2017/00199; A61B 2017/320064; A61B 2034/2065

(57) ABSTRACT

An ophthalmic surgical system includes an item of ophthalmic surgical equipment and one or more computing devices. The one or more computing devices are configured to receive a three-dimensional image of an eye of a patient; process the three-dimensional image using a machine learning model to obtain a density map of a crystalline lens of the eye of the patient; and control the item of surgical equipment according to the density map. The item of surgical equipment may be a treatment laser or phaco-vit tool. Aspiration pressure of the phaco-vit tool may be controlled based on a position of a distal end of the phaco-vit tool and a corresponding density in the density map.

### INTRODUCTION

The present disclosure relates generally to characterizing the density of cataracts for use in performing cataract surgery.

The human eye receives light through a clear outer portion called the cornea and focuses the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished light that is transmitted to the retina. This deficiency in the lens of the eye is medically known as a cataract. In addition, the crystalline lens may lose accommodation skills with age, which is called presbyopia. An accepted treatment for those conditions is the surgical removal of the crystalline lens followed by a replacement by an artificial intraocular lens (IOL).

### SUMMARY

In certain embodiments, an ophthalmic surgical system includes an item of ophthalmic surgical equipment and one or more computing devices. The one or more computing devices are configured to receive a three-dimensional image of an eye of a patient; process the three-dimensional image using a machine learning model to obtain a density map of a crystalline lens of the eye of the patient; and control the item of surgical equipment according to the density map. The item of surgical equipment may be a treatment laser or phaco-vit tool.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing ophthalmic treatments, in accordance with certain embodiments.

FIG. 2 illustrates the operation of a femtosecond laser for assisting in phacoemulsification.

FIG. 3 illustrates a process for performing phacoemulsification.

FIG. 4 illustrates the performance of three-dimensional imaging of an eye.

FIG. 5 illustrates a machine learning model for generating a density map of a crystalline lens in accordance with certain embodiments.

FIG. 6 illustrates a density map of a crystalline lens in accordance with certain embodiments.

FIG. 7 illustrates a process flow diagram of a method for performing femtolaser assisted cataract surgery (FLACS) using a density map in accordance with certain embodiments.

FIG. 8 illustrates a machine learning model for relating a density map and intra-operative history to outcomes in accordance with certain embodiments.

FIG. 9 is a process flow diagram of a method for predicting outcomes for cataract surgery using a machine learning model in accordance with certain embodiments.

FIG. 10 illustrates a machine learning model for determining diagnostic data for cataracts in accordance with certain embodiments.

FIG. 11 is a process flow diagram of a method for generating diagnostic data using a machine learning model in accordance with certain embodiments.

FIG. 12 illustrates an example computing device that implements, at least partly, one or more functionalities in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

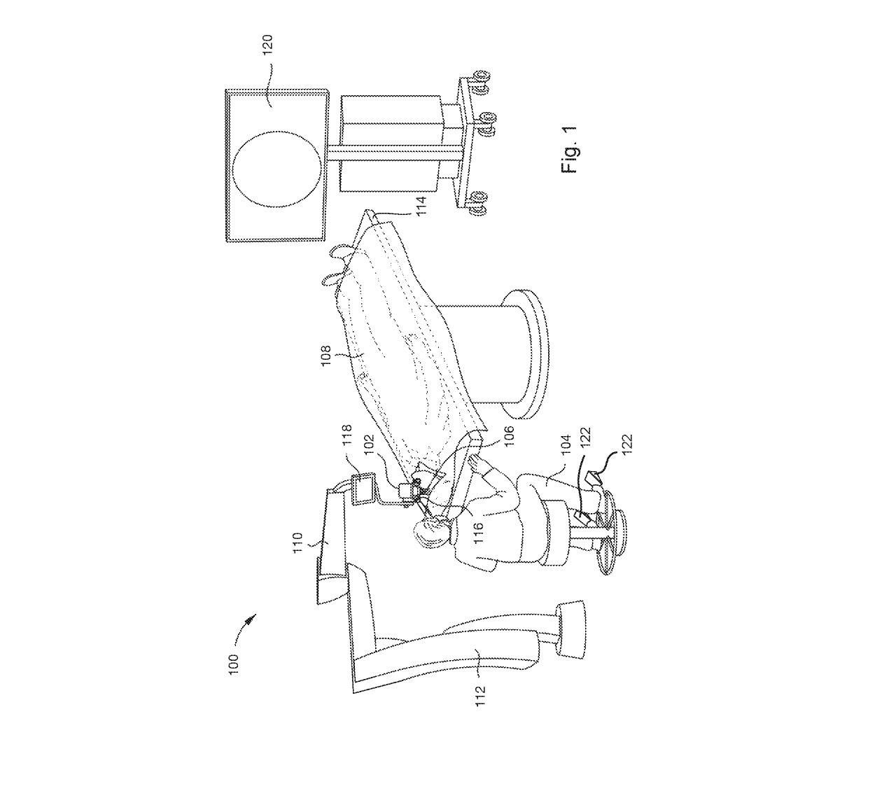

FIG. 1 illustrates an example operating environment including an ophthalmic surgical system 100 with which ophthalmic treatments may be performed. The ophthalmic surgical system 100 includes an ophthalmic microscope 102, used by a surgeon 104 to visualize structures on and in an eye 106 of a medical patient 108 in the field of view of the ophthalmic microscope 102. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and a broadband light source for capturing color (red, green, and blue) images and/or infrared images. The ophthalmic microscope 102 may, in certain embodiments, further include a multi-spectral imaging (MSI) device, and/or other type of imaging device. Digital images captured using the camera may be displayed on a display device within the ophthalmic microscope 102.

The ophthalmic microscope 102 may include two display devices that are viewable through binocular eyepieces 116 and that display images of the patient’s eye 106 captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively displayed on one or more display devices. For example, the one or more display devices may include a display device 118 fastened to the overhead arm 110 above the ophthalmic microscope 102.

In order to relieve the surgeon 104 from the need to constantly look into the eye pieces 116 to obtain a stereoscopic view, the one or more display devices may also include a display device 120 that can be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

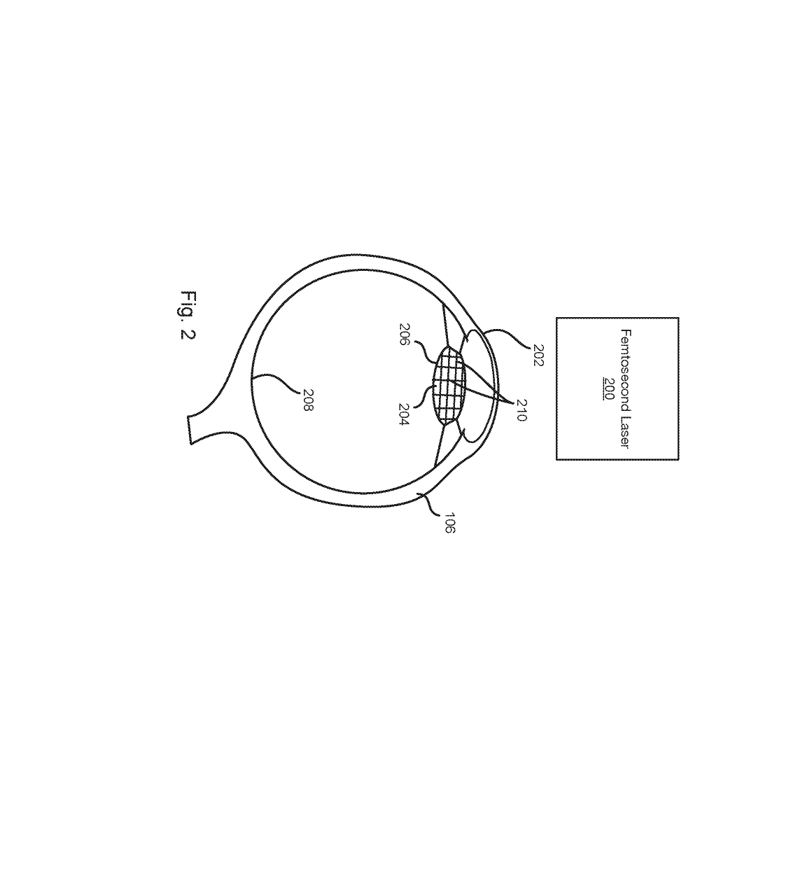

Referring to FIG. 2, in some embodiments, cataract surgery on the eye 106 may be facilitated by an item of surgical equipment, such as a laser 200, which may be a femtosecond laser 200, e.g., for performing femtosecond laser assisted cataract surgery (FLACS). The eye 106 includes the cornea 202 that directs light through the crystalline lens 204 contained within the capsular bag 206. The combined refraction of the cornea 202 and crystalline lens 204 focus light on the retina 208 to form an image.

Light from the femtosecond laser 200 may be directed through the cornea into the crystalline lens 204. A focal point of the laser may be scanned through various points within the crystalline lens 204 to disintegrate the crystalline lens 204 along section planes or contours 210. The disintegration may split the crystalline lens 204 into blocks or sheets or may instead create lines of weakness that soften the crystalline lens 204 to be more readily aspirated in a subsequent phacoemulsification step.

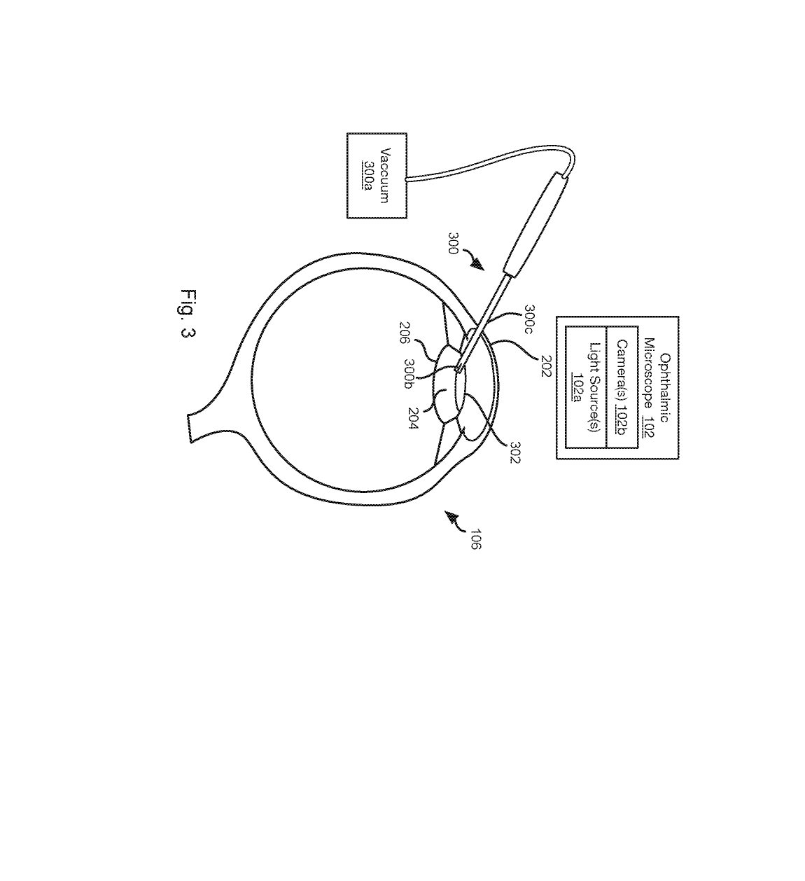

FIG. 3 illustrates the performance of the phacoemulsification step. An instrument may be inserted into the eye 106, such as through an incision formed at the limbus (the intersection of the cornea with the sclera (white) of the eye 106). The instrument may be used to create an opening 302 (“rhexis”) in the capsular bag 206. A phaco-vit tool 300 may then be inserted into the incision and into the capsular bag 206 in order to cut up and suction away the crystalline lens 204. The phaco-vit tool 300 may include an oscillating cutter 300b within a tube 300c that sucks in portions of the crystalline lens and cuts the portions, which are then sucked out through the tube 300c. The phaco-vit tool 300 may be coupled to a vacuum 300a supplying aspiration pressure to the phaco-vit tool 300. The vacuum 300a may be connected and disconnected from the tube 300c using a foot pedal 122. The amount of aspiration supplied by the vacuum 300a may also be controlled using a foot pedal 122. The speed of the oscillating cutter 300b may be pneumatically actuated and therefore change in correspondence with change in the aspiration pressure applied by the phaco-vit tool 300. The vacuum 300a may be an item of surgical equipment controlled based on a density map as described below.

The phacoemulsification procedure may be viewed by the surgeon 104 using the ophthalmic microscope 102. The ophthalmic microscope 102 may include one or more light sources 102a for illuminating the eye 106 and one or more cameras 102b for capturing images of the eye 106. The images may be displayed in real time on a display device 118, 120 or a display internal to the ophthalmic microscope 102.

Referring to FIG. 4, anatomy of the eye 106 may be visualized in three-dimensions, such as the illustrated X, Y, and Z directions that are mutually perpendicular. The Z direction may be defined as substantially (e.g., within 2 degrees of) parallel to the Z optical axis of the eye 106. The eye 106 may be visualized in section planes or other scanning pattern using optical coherence tomography (OCT) or a scanning laser ophthalmoscope (SLO) 400 (“OCT/SLO” 400). In particular, light scattered by the crystalline lens 204 may be measured using the OCT/SLO 400. In general, the amount of light scattered by a point within the crystalline lens 204 may corresponds to the density of the point, however this relationship is not exact. In addition, other artifacts are present due to tissue that light must pass through when traveling to and from that point.

Using the approaches described herein, the density of the crystalline lens may be determined accurately. Various uses for the density are also discussed in detail below.

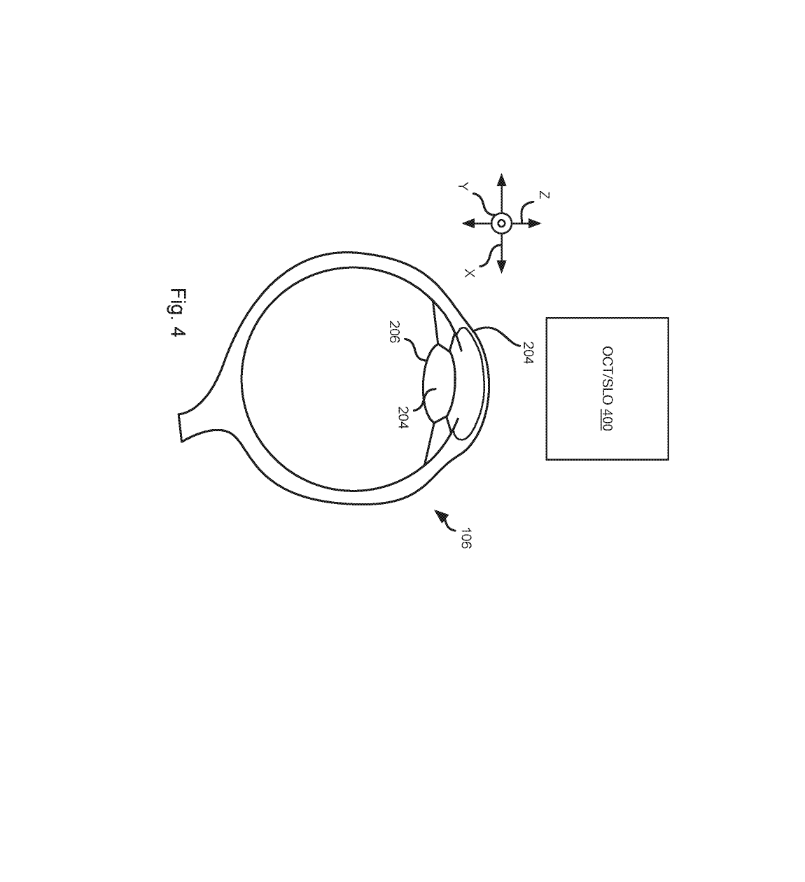

Referring to FIG. 5, a machine learning model 500 may be trained to generate a density map based on a three-dimensional image, such as a three-dimensional image output from the OCT/SLO 400. The machine learning model 500 may be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

The machine learning model 500 may be trained with training data entries 502 that include, as an input, a three-dimensional image 504, such as an image output by an OCT imaging device or SLO. In some embodiments, the machine learning model 500 may be trained for a specific type of imaging device (OCT or SLO) such that three-dimensional images 504 of all of the training data entries 502 will be captured using the same type of imaging device. In other embodiments, the three-dimensional images 504 may be captured using multiple types of imaging devices.

Each training data entry 502 may include, as a desired output, a density map 506. The density map 506 records the density at a plurality of positions within a crystalline lens 204 that was imaged by the three-dimensional image 504 of the training data entry 502. The density map may be obtained in various ways. In one approach, cadaver crystalline lenses 204 may be imaged and then dissected to measure density throughout the crystalline lenses 204. In another approach, a crystalline lens 204 may be imaged prior to phacoemulsification. The density of the crystalline lens 204 undergoing phacoemulsification may be estimated based on the amount of time spent by a phaco-vit tool 300 at each of a plurality of points within the crystalline lens 204, e.g., the estimated density increasing with increasing time.

The machine learning model 500 may process the three-dimensional image 504 of each training data entry 502 to obtain an estimated density map. A training algorithm 508 may compare the estimated density map to the density map 506 of the training data entry 502 and update one or more parameters of the machine learning model 500 according to the comparison such that the machine learning model 500 is trained to generate a density map for a given three-dimensional image.

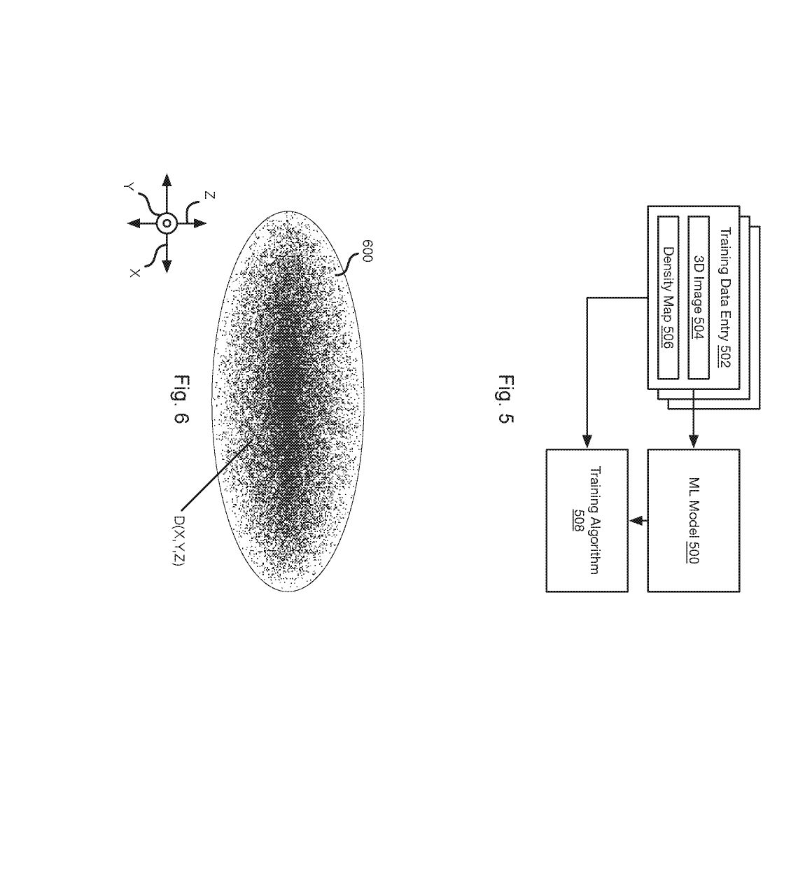

Referring to FIG. 6, the output of the machine learning model 500, and the density map 506 of each training data entry 502, may include a density value (D(X,Y,Z)) that either corresponds to or can be mapped to at least one volumetric pixel (voxel) (V(X,Y,Z) of the three-dimensional image 504. The density map 506 may have the same resolution or a higher or lower resolution than the three-dimensional image 504. A polar coordinate system or other representation of the volume of the crystalline lens 204 may also be used to define each voxel in the density map. The density map may also be represented as a density function of X, Y, and Z.

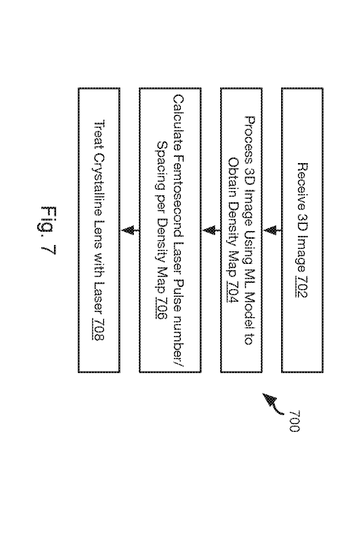

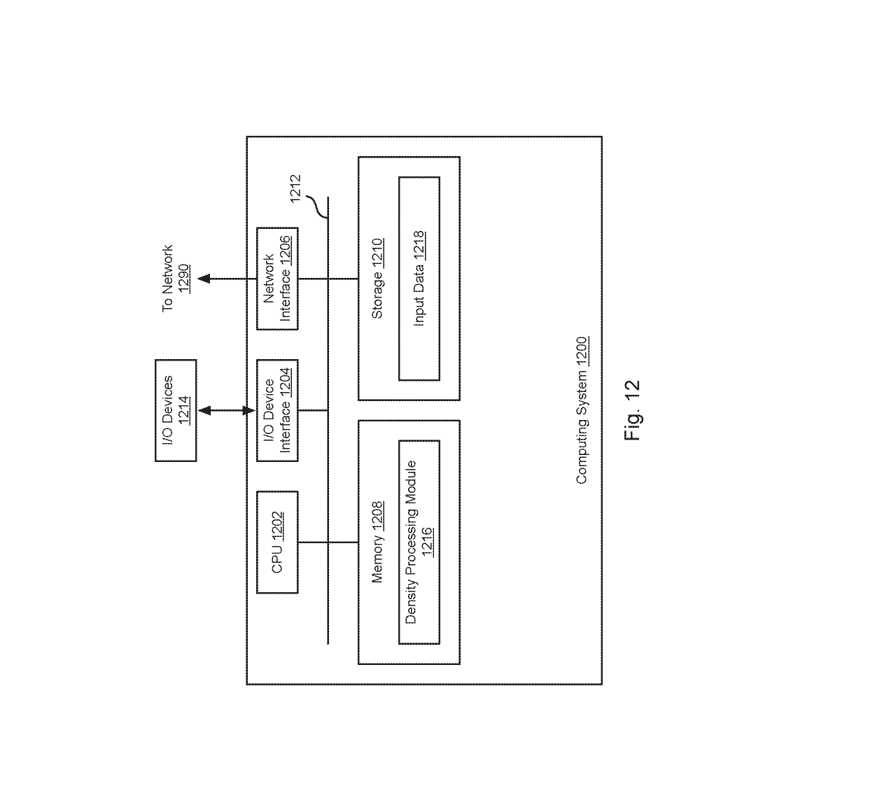

FIG. 7 illustrates method 700 that may use a density map 600 determined using the machine learning model 500. The method 700 may be performed using the computing system 1200 of FIG. 12. The method 700 may include receiving, at step 702, a three-dimensional image from the OCT/SLO 400. For example, the three-dimensional image may be obtained pre-operatively. The three-dimensional image may be created using whichever type of imaging device was used to train the machine learning model 500 in the case of a machine learning model 500 trained with three-dimensional images 504 from a single type of imaging device.

The method 700 may include processing, at step 704, the three-dimensional image using the machine learning model 500 to obtain a density map 600. The method 700 may include calculating, at step 706, parameters for controlling the laser 200 according to the density map. For example, the parameters may include the count of pulses directed at a given three-dimensional position within the crystalline lens 204, a duration of pulses. The parameters may include the spacing of points within the crystalline lens 204 that will be targeted using the laser 200. For example, the focal point may be scanned across a three dimensional pattern (e.g., raster pattern, pie segments, spiral, etc.) within the crystalline lens 204. The speed of movement of the focal point over the pattern may be inversely related to density: a slower scanning speed in denser regions than in less dense regions. In some embodiments, the parameters may include the duration and/or intensity of pulses directed at a particular point within the crystalline lens 204.

For example, a first point in the crystalline lens having a first corresponding density in the density map may receive a first pulse number and have a first spacing relative to adjacent points that are targeted with the laser 200. A second point in the crystalline lens having a second corresponding density in the density map that is less than the first corresponding density may receive a second pulse number that is less than the first pulse number. The second spacing relative to adjacent points that are targeted with the laser 200 that may additionally or alternatively be less than the first spacing.

The method 700 may then include treating, at step 708, the crystalline lens 204 with the laser 200. In particular, each point selected at step 706 may receive the number of pulses selected at step 706.

Using the method 700, more dense parts of the crystalline lens 204 will be more strongly disintegrated and/or disintegrated into smaller blocks or section planes than less dense parts. The crystalline lens 204 will therefore be more easily cut and aspirated during a subsequent phacoemulsification step, which will reduce the time spent performing phacoemulsification relative to prior approaches. In addition, the amount of laser light emitted into the eye 106 can be tuned to be more precisely that which is required to facilitate phacoemulsification, as opposed to light generated using constant parameters selected for a maximum density, average density, or other single value used to characterize the density of the crystalline lens 204. Phototoxicity may therefore be reduced.

The method 700 may be performed using multiple computing devices, any of which may be a computer system having some or all of the attributes of the computer system 1200. For example, the computing device that generates the density map at step 704 may be the same as or different from the computing device that performs steps 706 and/or step 708.

Referring to FIG. 8, the illustrated machine learning model 800 may be used to guide a phacoemulsification step whether performed with or without softening using the laser 200. The machine learning model 800 may be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

The machine learning model 800 may be trained with training data entries 802 that include, as an input, a three-dimensional image 804, such as an image output by an OCT imaging device or SLO. In some embodiments, the machine learning model 800 may be trained for a specific type of imaging device (OCT or SLO) such that three-dimensional images 804 of all of the training data entries 802 will be captured using the same type of imaging device. In other embodiments, the three-dimensional images 804 may be captured using multiple types of imaging devices.

Each training data entry 802 may include, as a desired output, a density map 806. The density map 806 records the density at a plurality of positions within a crystalline lens 204 that was imaged by the three-dimensional image 804 of the training data entry 802. The density map may be obtained using any of the approaches described above.

The training data entry 802 may include other information such as intra-operative data 808 and an outcome 810. The intra-operative data may include information describing a phacoemulsification step such as values for parameters describing the phacoemulsification step over time. The parameters may include aspiration pressure over time, a position of a tip of a phaco-vit tool 300 over time, or one or more other parameters. The intra-operative data may include video and/or three-dimensional images captured using the ophthalmic microscope 102 during the phaco-emulsification step or other stages of the cataract surgery. The intra-operative data may additionally include one or more items of pre-operative data describing the eye of the patient. Alternatively, the density map 806 is the only pre-operative data used.

The outcome 810 may include some metric of success of cataract surgery, such as duration of the cataract surgery, a metric describing aspiration (e.g., volume aspirated, integral of aspiration over time, or other cumulative metrics of aspiration, energy used by the phaco-vit tool 300), intraocular pressure (IOP), endothelial cell loss, distance visual acuity, near point, a combination of distance acuity and near point, or other metric of success of a cataract surgery.

In some embodiments, as discussed below, an outcome 810 may be estimated at various time points in a cataract surgery. Accordingly, multiple training data entries 802 may be obtained for a single procedure, each training data entry 802 including a different amount of the intra-operative data. For example, a first training data entry 802 may include intra-operative data recorded from a starting time to a first intermediate time prior an end time, a second training data entry 802 may include intra-operative data recorded from the starting time to second first intermediate time prior to the end time and after the first intermediate time, and a third training data entry 802 may include intra-operative data recorded from the starting time to the end time. There may be any number of training data entries 802 generated for the same cataract surgery each corresponding to a different portion of the cataract surgery. The start time and end time may be the start and end time of the entire cataract surgery or the start and end time of just the phaco-emulsification step.

The machine learning model 800 may process the three-dimensional image 804 each training data entry 802 to obtain an estimated density map. A training algorithm 812 may compare the estimated density map to the density map 806 of the training data entry 802 and update one or more parameters of the machine learning model 800 according to the comparison such that the machine learning model 800 is trained to generate a density map for a given three-dimensional image.

The machine learning model 800 may additionally receive, as an input, the intra-operative data 808 of the training data entry 802 and one or both of the three-dimensional image 804 of the training data entry 802 and the density map as estimated by the machine learning model 800 for the training data entry 802. The result of the processing may be an estimated outcome. The training algorithm 812 may compare the estimated outcome to the outcome 810 of the training data entry 802 and update the machine learning model 800 according to the comparison.

The machine learning model 800 may be composed of multiple machine learning models, including one trained to estimate the density map as described above and another trained to estimate the outcome as described above.

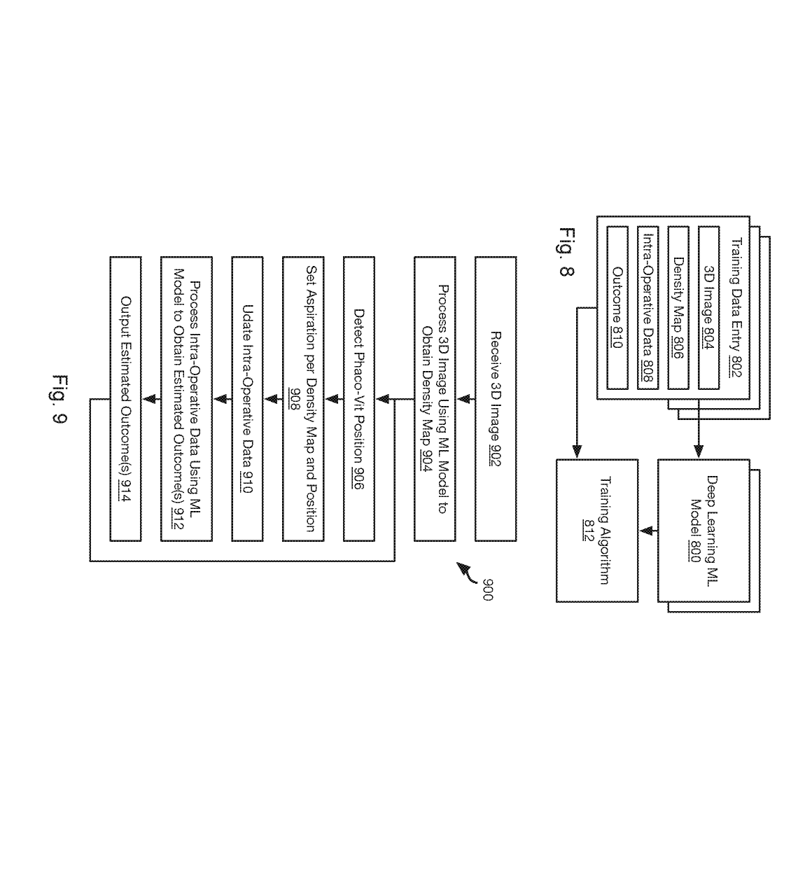

FIG. 9 illustrates method 900 that may use a density map 600 determined using the machine learning model 800. The method 900 may be performed using the computing system 1200 of FIG. 12. The method 900 may include receiving, at step 902, a three-dimensional image from the OCT/SLO 400. For example, the three-dimensional image may be obtained pre-operatively. The three-dimensional image may be created using whichever type of imaging device was used to train the machine learning model 800 in the case of a machine learning model 800 trained with three-dimensional images 804 from a single type of imaging device. The method 900 may include processing, at step 904, the three-dimensional image using the machine learning model 800 to obtain a density map 600. Steps 902 and 904 may be performed pre-operatively. The remaining steps may be performed intra-operatively.

The method 900 may include detecting, at step 906, a position of a distal end of a phaco-vit tool 300. Step 906 may include detecting the distal end of the phaco-vit tool 300 in an output of one or more cameras of the ophthalmic microscope 102. Where the ophthalmic microscope 102 is a binocular ophthalmic microscope, step 906 may include determining a three-dimensional position of the distal end of the phaco-vit tool 300 from a pair of binocular images.

The method 900 may include setting, at step 908, the aspiration pressure of the phaco-vit tool 300 according to the density map and the position detected at step 906. For example, the aspiration pressure may be set according to a function that increases with increasing density at a point in the density map corresponding to the position, such as according to a linear relationship or other experimentally determined relationship between aspiration pressure and density. Other parameters may also be varied, such as an oscillating speed of the phaco-vit tool, such as according to a function that increases oscillating speed with increasing density as recorded in the density map.

Relating of the position to the coordinate system of the density map may be performed using a registration step by which images from the ophthalmic microscope are registered with respect to one or more pre-operative images of the eye 106.

In some embodiments, step 908 may be supplemented with or replaced with displaying information from the density map to the surgeon 104, such as on one of the display devices 118, 120 or on a display device internal to the ophthalmic microscope 102. For example, a density corresponding to the detected position of the distal end of the phaco-vit tool 300 may be displayed in the form of a numerical value or other graphical representation of the density.

The method 900 may include updating, at step 910, intra-operative data. For example, step 910 may include updating a record of intra-operative data for the cataract surgery being performed since a previous iteration of step 910. The intra-operative data may include any of the information described above as possibly being included in the intra-operative data 808. The intra-operative data may include video and/or three-dimensional images captured using the ophthalmic microscope 102 during the cataract surgery or data derived therefrom, such as a position of a distal end of a phaco-vit tool 300.

The method 900 may include processing, at step 912, the intra-operative data and one or both of the three-dimensional images from step 902 and the density map from step 904 using the machine learning model 800 to obtain an estimated outcome. The estimated outcome may be output at step 914, such as on a display device 118, 120 or a display device internal to the ophthalmic microscope 102.

In some embodiments, steps 910-914 may be omitted and the method 900 is used exclusively to control the operation of the phaco-vit tool 300. In some embodiments, an output is also displayed indicating a recommended dwell time for a current position of the distal end of the phaco-vit tool 300. For example, the dwell time for a position may be inversely proportional to the density corresponding to the position in the density map.

The method 900 may be performed using multiple computing devices, any of which may be a computer system having some or all of the attributes of the computer system 1200. For example, the computing device that generates the density map at step 904 may be the same as or different from the computing device that performs the remaining steps of the method 900.

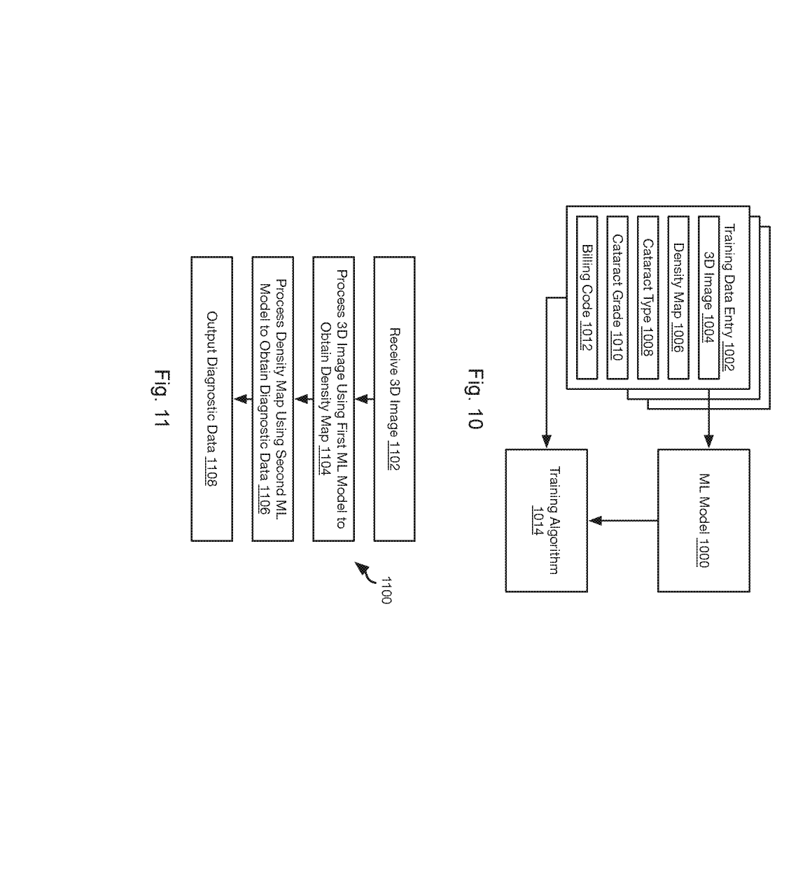

FIG. 10 illustrates a machine learning model 1000 to obtain diagnostic data for use in a cataract surgery. In particular, the machine learning model 1000 may be used preoperatively. The machine learning model 1000 may be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

The machine learning model 1000 may be trained with training data entries 1002 that include, as an input, a three-dimensional image 1004, such as an image output by an OCT imaging device or SLO. In some embodiments, the machine learning model 1000 may be trained for a specific type of imaging device (OCT or SLO) such that three-dimensional images 1004 of all of the training data entries 1002 will be captured using the same type of imaging device. In other embodiments, the three-dimensional images 1004 may be captured using multiple types of imaging devices.

Each training data entry 1002 may include, as another input, a density map 1006. The density map 1006 records the density at a plurality of positions within a crystalline lens 204 that was imaged by the three-dimensional image 1004 of the training data entry 1002. The density map may be obtained using any of the approaches described above and may also be generated using a machine learning model, such as the machine learning model 500 described above.

In some embodiments, the three-dimensional image 1004 is omitted and only the density map 1006 is included. In other embodiments, the density map 1006 is omitted and only the three-dimensional image 1004 is included. In some embodiments, only the three-dimensional image 1004 is included and a density map 1006 is generated from the three-dimensional image 1004 during training of the machine learning model 1000.

Each training data entry 1002 may include one or more items of diagnostic data. For example, the one or more items of diagnostic data may include a cataract type 1008. The type may indicate whether a cataract represented by the three-dimensional image 1004 and density map 1006 is nuclear, cortical, posterior subcapsular, anterior subcapsular, diabetic snowflake, posterior polar, traumatic, congenital, polychromatic, or of some other type.

The diagnostic data may include a grade 1010 of the cataract represented by the three-dimensional image 1004 and density map 1006. For example, the grade 1010 may be a classification according to the lens opacities classification system III (LOCS III).

The diagnostic data may include a billing code 1012. The billing code 1012 may represent a cost of a cataract surgery for the cataract represented by the three-dimensional image 1004 and density map 1006. The cost may be a function of the density of the crystalline lens 204 and intra-operative complications that occurred during cataract surgery performed on the eye represented in the three-dimensional image 1004.

Some or all of the items of diagnostic data may be assigned by a human operator. Some or all of the items of diagnostic data may be assigned by a human post-operatively using information obtained during the cataract surgery. For example, intra-operative complications may be discovered during the cataract surgery. The type or grade of the cataract may be determined based on observation during the cataract surgery.

The machine learning model 1000 may process the three-dimensional image 1004 and density map 1006 of each training data entry to obtain estimated diagnostic data. A training algorithm 1014 may compare the estimated diagnostic data to the diagnostic data of the training data entry 1002 and update the machine learning model 1000 according to the comparison. In other embodiments, the machine learning model 1000 processes only the three-dimensional image 1004. In such embodiments, the machine learning model 1000 may either omit any processing of a density map 1006 or may be trained to generate the density map 1006 using one stage that is then processed in a subsequent stage. In some embodiments, the machine learning model 1000 processes only the density map 1006 and processing of the three-dimensional image 1004 is omitted.

Various modifications of the machine learning model 1000 and associated training data entries 1002 may be implemented. For example, the training data entry 1002 for an eye of a patient may include other patient data, such as any diagnosis with diabetes, patient-reported vision problems, age, gender, or other data.

FIG. 11 illustrates a method 1100 that uses the machine learning model 1000. The method 1100 may be performed using the computing system 1200 of FIG. 12. The method 1100 may include receiving, at step 1102, a three-dimensional image from the OCT/SLO 400, the three-dimensional image including a representation of an eye of a patient. For example, the three-dimensional image may be obtained pre-operatively. The three-dimensional image may be created using whichever type of imaging device was used to train the machine learning model 1000 in the case of a machine learning model 1000 trained with three-dimensional images 1004 from a single type of imaging device.

The method 1100 may include processing, at step 1104, the three-dimensional image using a first machine learning model, e.g., the machine learning model 500 to obtain a density map 600. The method 1100 may include processing, at step 1106, the density map 600 using a second machine learning model, e.g., the machine learning model 1000, to obtain diagnostic data. Step 1106 may include processing any other items of data listed above as possibly being included in a training data entry 1002, such as the three-dimensional image 1004 and/or one or more items of patient information. As also noted above, step 1106 may include processing only a three-dimensional image and omitting processing of a density map.

The method 1100 may include outputting, at step 1108, the diagnostic data. The diagnostic data may be stored in a database, sent in an email, added to a medical record of the patient, or output to a display device.

The method 1100 may be performed using multiple computing devices, any of which may be a computer systems having some or all of the attributes of the computer system 1200. For example, the computing device that generates the density map at step 1104 may be the same as or different from the computing device that performs the remaining steps of the method 1100.

FIG. 12 illustrates an example computing system 1200. The ophthalmic microscope 102, display devices 118, 120 may incorporate a computing device having some or all of the attributes of the computing system 1200.

As shown, computing system 1200 includes a central processing unit (CPU) 1202, one or more I/O device interfaces 1204, which may allow for the connection of various I/O devices 1214 (e.g., keyboards, displays, mouse devices, pen input, etc.) to computing system 1200, network interface 1206 through which computing system 1200 is connected to network 1290, a memory 1208, storage 1210, and an interconnect 1212.

CPU 1202 may retrieve and execute programming instructions stored in the memory 1208. Similarly, CPU 1202 may retrieve and store application data residing in the memory 1208. The interconnect 1212 transmits programming instructions and application data, among CPU 1202, I/O device interface 1204, network interface 1206, memory 1208, and storage 1210. CPU 1202 is included to be representative of a single CPU, multiple CPUs, a single CPU having multiple processing cores, and the like.

Memory 1208 is representative of a volatile memory, such as a random access memory, and/or a nonvolatile memory, such as nonvolatile random access memory, phase change random access memory, or the like. As shown, memory 1208 may store executable code implementing a density processing module 1216, which may implement some or all of the methods 700, 900, 1100 described above.

Storage 1210 may be non-volatile memory, such as a disk drive, solid state drive, or a collection of storage devices distributed across multiple storage systems. The storage 1210 may store input data 1218 collected and processed according to some or all of the methods 700, 900, 1100, such as any of the items of data described above as being part of training data entries 502, 802, 1002 used to train and utilize a machine learning model 500, 800, 1000.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic surgical system comprising:
an item of ophthalmic surgical equipment; and
one or more computing devices configured to:
receive a three-dimensional image of an eye of a patient;
process the three-dimensional image using a machine learning model to obtain a density map of a crystalline lens of the eye of the patient; and
control the item of ophthalmic surgical equipment according to the density map.

2. The ophthalmic surgical system of claim 1, wherein the three-dimensional image is a scanning laser ophthalmoscope image.

3. The ophthalmic surgical system of claim 2, wherein the three-dimensional image is an optical coherence tomography (OCT) image.

4. The ophthalmic surgical system of claim 1, wherein the item of surgical equipment is a phaco-vit tool.

5. The ophthalmic surgical system of claim 4, wherein the one or more computing devices are configured to control an aspiration pressure of the phaco-vit tool during an ophthalmic treatment according to the density map.

6. The ophthalmic surgical system of claim 5, wherein the one or more computing devices are configured to:
receive one or more images from an ophthalmic microscope during an ophthalmic treatment;
detect a position of a distal end of the phaco-vit tool in the one or more images; and
set the aspiration pressure according to a density in the density map at a point corresponding to the position of the distal end of the phaco-vit tool.

7. The ophthalmic surgical system of claim 5, wherein the one or more computing devices are configured to:
receive one or more images from an ophthalmic microscope;
detect a position of a distal end of the phaco-vit tool in the one or more images; and
set an oscillating speed of a cutter of the phaco-vit tool according to a density in the density map at a point corresponding to the position of the distal end of the phaco-vit tool.

8. The ophthalmic surgical system of claim 5, wherein the one or more computing devices are configured to:
receive intra-operative data during the ophthalmic treatment;
process the intra-operative data with the machine learning model to obtain an estimated outcome; and
display the estimated outcome on a display device during the ophthalmic treatment.

9. The ophthalmic surgical system of claim 8, wherein the estimated outcome is at least one of a metric of aspiration using the phaco-vit tool.

10. The ophthalmic surgical system of claim 8, wherein the estimated outcome is at least one of intraocular pressure (IOP), endothelial cell loss, distance vision acuity, and near point.

11. A method comprising:
receiving, by a computer system, a three-dimensional image of an eye of a patient;
processing, by the computer system, the three-dimensional image using a machine learning model to obtain a density map of a crystalline lens of the eye of the patient; and
controlling, by the computer system, an item of surgical equipment according to the density map.

12. The method of claim 11, wherein the three-dimensional image is a scanning laser ophthalmoscope image.

13. The method of claim 12, wherein the three-dimensional image is an optical coherence tomography (OCT) image.

14. The method of claim 11, wherein the item of surgical equipment is a phaco-vit tool.

15. The method of claim 14, wherein controlling the item of surgical equipment according to the density map comprising controlling an aspiration pressure of the phaco-vit tool during an ophthalmic treatment according to the density map.

16. The method of claim 15, further comprising:
receiving, by the computer system, one or more images from an ophthalmic microscope during an ophthalmic treatment;
detecting, by the computer system, a position of a distal end of the phaco-vit tool in the one or more images; and
setting, by the computer system, the aspiration pressure according to a density in the density map at a point corresponding to the position of the distal end of the phaco-vit tool.

17. The method of claim 15, further comprising:
receiving, by the computer system, one or more images from an ophthalmic microscope;
detecting, by the computer system, a position of a distal end of the phaco-vit tool in the one or more images; and
setting, by the computer system, an oscillating speed of a cutter of the phaco-vit tool according to a density in the density map at a point corresponding to the position of the distal end of the phaco-vit tool.

18. The method of claim 15, further comprising:
receiving, by the computer system, intra-operative data during the ophthalmic treatment;
processing, by the computer system, the intra-operative data with the machine learning model to obtain an estimated outcome; and
displaying, by the computer system, the estimated outcome on a display device during the ophthalmic treatment.

19. The method of claim 18, wherein the estimated outcome is at least one of a metric of aspiration using the phaco-vit tool.

20. The method of claim 18, wherein the estimated outcome is at least one of intraocular pressure (IOP), endothelial cell loss, distance vision acuity, and near point.
